SEQUENCE LISTING (2 sequences)

SEQ ID NO: 1 - DNA, 23 bp, Artificial Sequence
  Chemically synthesized primer
ttcttgaaggaccaaccgagagg

SEQ ID NO: 2 - DNA, 25 bp, Artificial Sequence
  Chemically synthesized primer
gctaacgcaggtaagatttccaagc

(19) United States
(12) Patent Application Publication
(10) Pub. No.: US 2007/0134668 A1
(43) Pub. Date: Jun. 14, 2007

(54) Method for analysis by molecular hybridization of nucleic acids and kit for carrying out said method

(76) Inventors: Olivier Dutrecq, Rue de la Croix-Rouge (BE); Sophie Roussel, Route d'Ohain (BE); Philippe Lepoivre, Rue de Perwez (BE); Jean Kummert, Rue du Centre (BE); Mohammed Haissam Jijakli, Chaussee de Waterloo (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/579,674

(22) PCT Filed: Nov. 18, 2004
(86) PCT No.: PCT/EP04/13220

§ 371 Date: May 18, 2006

(30) Foreign Application Priority Data
Nov. 3, 2003 (FR) 0312863

(51) Int. Cl. C12Q 1/68; C12N 1/08

(52) U.S. Cl. 435/6; 435/270

(57) ABSTRACT

The invention concerns a method of assaying by molecular hybridization to detect the presence of nucleic acids that includes a step of acquiring samples of biological material by a sampling device with abrasive sampling means capable of retaining biological material in the form of cells, and a kit for application of this method.

The present invention concerns an assaying method to detect the presence of nucleic acids by molecular hybridization and a kit for applying this method.

Among the methods for assaying nucleic acids by molecular hybridization, PCR (polymerase chain reaction) is being increasingly widely used. It is preferred over immunological assay methods, such as the ELISA, because of its greater sensitivity.

Furthermore, PCR allows one to develop targeted methods of very great specificity, depending on the purpose of the assay.

Even so, certain specific constraints are involved in the use of PCR. A major group of such constraints involves protocols for preparing the specimens that are being assayed.

As a matter of fact, biological materials may contain substances liable to interfere with the PCR amplification process.

Among such substances having an inhibitory effect on PCR, one can mention hemoglobin (human and animal) and the polysaccharides and phenolic compounds in plants.

Fresh tissue is likely to contain sizeable quantities of such inhibitors. Furthermore, oxidation effects which take place after such tissues are sampled will augment the inhibitory effect of these substances.

To prevent the oxidizing of tissues and the presence of inhibitors as much as possible during PCR assays, one customarily uses techniques for extracting and purifying the target nucleic acids, so as to assure an optimal PCR reaction.

Thus, one can extract nucleic acids and then purify them by various techniques, such as purification by affinity, filtration on gel or precipitation by isopropanol/ethanol.

These extraction and purification techniques may take up to several days, and they are costly.

There has thus been a progressive turning to simpler assay protocols that do not require further purification of the nucleic acids.

In one method of the prior art, the assay for nucleic acids is done on a crude extract of pulverized biological material. The sampling involves taking tissue for assay from fresh or stabilized material (using either lyophilization or freezing) and in rather substantial quantities (200-400 mg). The tissue samples should be quickly put into the grinding buffer. It is likewise essential to keep them at a low temperature as much as possible. The tissue is pulverized in a manual or automatic grinder, which produces the crude, ground extract. One centrifuges this crude extract and recovers the supernatant solution for the assay. One can thus carry out this assay by molecular hybridization of nucleic acids by starting with a representative extract of the totality of nucleic acids of the biological material sampled.

Unfortunately, this technique has the drawback that when the tissues are fragmented outside of the buffer prior to the grinding, the oxidation of the inhibitors which are widespread in the tissues (such as phenolic compounds in the case of plant tissues) considerably enhances the inhibitory properties on the PCR of the crude extract obtained. This is all the more troublesome when there is no purification step provided for the nucleic acids, since the work is done directly from this crude extract. Moreover, the enzymatic reactions of the RNases and DNases involved may likewise impair the PCR reaction by deteriorating the target nucleic acids.

According to this technique, one needs to work quickly on a large quantity of fresh biological material, or alternatively take energy and time consuming steps such as lyophilization or freezing to preserve the samples.

It has been proposed, e.g., in EP-A-0 444 649, to take a sample of plant material on an adsorbent membrane which one presses against a fresh tissue. Such a membrane can, for example, be made of nitrocellulose, nylon, or modified nylon. The nucleic acids should be fixed on the membrane. However, this technique remains marginal and little used, since it raises problems in terms of reliability.

It thus remains desirable to find a simple method for assaying nucleic acids by hybridization, making it possible to obtain results that are just as reliable as those of the prior art, even though there is no purification step for the nucleic acids. It is likewise desirable to propose a method that makes it possible to work with the samples of biological material in lower quantity as compared to those traditionally used, and to obtain a good preservation of the samples without resorting to complicated measures.

Now, it has been discovered, surprisingly, that it is possible to obtain such a result by the invention, making use of a method of assaying by molecular hybridization of nucleic acids according to the invention, which comprises a step of taking samples of biological material by a sampling device comprising abrasive sampling means capable of retaining the biological material in the form of cells.

By “abrasive sampling means” is meant means able to penetrate, by the effect of pressure, into the surface of the biological material and produce an incision therein, so that biological material in the form of cells is retained by said means.

As will be shown below, it has been found that the samples taken by these abrasive sampling means are very quickly dehydrated in the surrounding air, do not become oxidized, and can be kept for several weeks. It has also been found that the assay results that are obtained are just as reliable several weeks after the sampling, as when the assay is performed at once.

By “molecular hybridization” is meant, in the context of the present text, any reaction of pairing of two single-strand nucleic acid molecules whose sequences are complementary to form a stable double-strand molecule.

By “PCR” or “polymerase chain reaction” is meant, in the context of the present text, any molecular hybridization technique able to produce a large number of specific DNA sequences from a complex genome. PCR makes it possible to amplify the target DNA or RNA and to detect it by staining techniques, fluorescent or radioactive labeling. Non-limiting examples thereof are IC-PCR, RT-PCR, and real time PCR.

By “biological material” is meant, in the context of the present text, any material of living origin: animal, plant, human, eucaryotic or procaryotic.

Preferably, sampling of biological material by the method according to the invention is done in the surrounding air. One works in an atmosphere unsaturated by humidity, preferably a dry atmosphere.

One can simply leave the abrasive means, loaded with their respective samples, under the effect of this surrounding air, which will allow the sample of biological material to dry out and become dehydrated. This measure is sufficient to guarantee a good preservation of the samples. This is very advantageous, because one does not have to proceed at once with the assaying of nucleic acids by hybridization.

One can also intensify or accelerate the drying process by using supplemental drying means, such as a device blowing cold or hot air (hair dryer, etc.), or by increasing the temperature (moving along the top of a radiator, for example).

Thanks to the method of the invention, sampling can be done outside of a laboratory where the assaying is to take place. In this case, the method includes a transport phase of the abrasive sampling means loaded with their respective samples of biological material to said laboratory.

For sampling biological material of plant origin, one can thus work out in the field without worrying about possible degradation of the samples, which is not so with other methods of the prior art, which require difficult preservation steps such as freezing or lyophilization of the samples.

Samples of biological material received at the laboratory by the abrasive sampling means can be processed as soon as they are received, or according to the needs and desires of the person performing the assay.

The method according to the invention can also include an extraction step of the nucleic acids, comprising a step of immersion of the abrasive sampling means loaded with their respective samples of biological material into an extraction buffer, a step of agitation in the extraction buffer, a separation step, and a step for recovering clarified solution containing the nucleic acids. This separation step preferably consists of a centrifugation, with the supernatant comprising the clarified solution.

Preferably, assaying by molecular hybridization is done by polymerase chain reaction (PCR).

Assaying nucleic acids by molecular hybridization according to the method of the invention can be done in order to determine the presence of a pathogenic agent in the biological material. The biological material consists of material of plant origin. The invention therefore has one of its applications in the area of diagnosis of plant diseases.

The invention also concerns a kit for implementing the invention's assaying method. This kit includes a sampling device, comprising abrasive sampling means capable of retaining biological material in the form of cells.

These sampling means may include a solid material having an abrasive outer surface, for example, silica, glass, metals, carbon fibers and plastics or any other suitable material, as well as mixtures of these materials. Preferably, the abrasive outer surface has a roughness suitable for retaining the cells of biological material, such as points, hooks, or hairs. Hard hairbrushes are suitable as the abrasive sampling means in the sense of the present text.

The abrasive sampling means of the device for taking samples of biological material in the kit according to the invention can be rigid or flexible (paper or glass), or alternatively the sampling device can comprise a support suitable for holding the abrasive sampling means.

The kit comprises means for the transport of the abrasive sampling means, for example, a pouch having small compartments for individually placing the samples dried according to the invention on the abrasive sampling means.

The kit preferably comprises means of identification allowing one to trace the origin of the samples. These means can take any adequate form (inscriptions, bar code), and be placed on the transport means or on the support or in certain cases even on the abrasive sampling means (for example, on the back of a piece of sandpaper).

The kits as described above are intended for the sample takers, who do not necessarily themselves perform the assaying of nucleic acids by molecular hybridization.

The invention also involves kits for the persons performing such an assay. The kit then comprises various reagents needed for this assay, possibly some extraction buffer for assaying nucleic acids by hybridization, and any necessary reagents, such as the specific reagents of the PCR reactions.

Thus, for example, the kit may also comprise the reagents needed for universal assays by RT-PCR, such as: the Kit Titan One Tube RT-PCR (Roche) as well as the rules to follow to define the amplification primers and specific probes to be included according to the needs in this optimized protocol.

It may also comprise the reagents needed for specific PCR assays for the detection of one or more pathogenic agents in plant material, namely: the pairs of primers targeting a pathogen or a combination of pathogens, such as:
PNRSV 10F and PNRSV 10R for detection of Prunus Necrotic Ringspot Virus,
ASGV 5F and ASGV 5R for detection of the Apple Stem Grooving Virus,
PDV 17F and PDV 12R for detection of the Prune Dwarf Virus,
ASPV 4F and ASPV 4R for detection of the Apple Stem Pitting Virus,
ApMV 1F and ApMV 1R for detection of the Apple Mosaïc Virus,
ACLSV 5F and ACLSV 8R for detection of the Apple Chlorotic Leaf Spot Virus,

The invention shall be illustrated below by describing sample embodiments, making reference to the enclosed drawings, in which

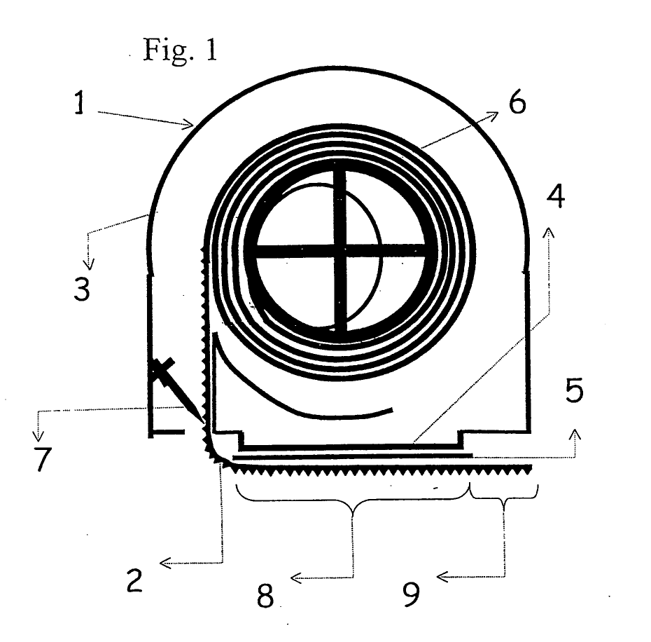

FIG. 1 is a cross section view of a first embodiment of a sampling device designed to implement a method according to the invention;

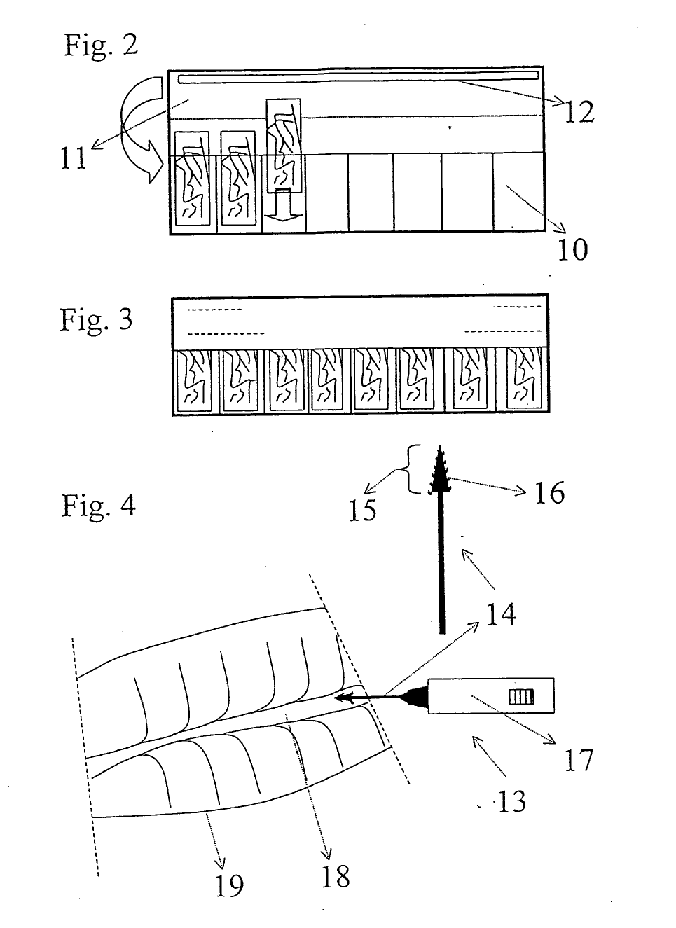

FIGS. 2 and 3 illustrate a means for sorting and transport of samples taken thanks to the sampling device illustrated in FIG. 1, and

FIG. 4 shows a second embodiment of a sampling device designed to implement a method according to the invention.

### EXAMPLES

### Example 1

### Detection of the Virus PNRSV (Prunus necrotic Ringspot Virus) in Various Branches of Infected Cherry Trees by a RT-PCR on Crude Extract

One performs an assay of nucleic acids by hybridization according to the invention by taking samples, during dry weather, of branches of fruit trees for sending to the laboratory to detect the presence of a phytopathogenic agent by RT-PCR assay.

In this sample embodiment, one uses a sampling device 1 as shown in FIG. 1. The sandpaper 2, marketed under the brand S.A.M. Corindon Extra Grain 60, is integrated in a casing 3 which can easily be held in the hand and presents a plane surface 4 on which the sandpaper 2 is secured by means of a fixation device 5, which can be an adhesive tape, a Velcro strip, or any other system of fixation. The casing 3 contains a spool 6, making it possible to release new surfaces of untouched sandpaper as the sampling process continues, after the blade 7 has cut off the sandpaper loaded with the sample of biological material. The fresh sandpaper is placed on an area for taking samples 8. An area 9 that is not covered by samples of biological material is provided at the end of the sandpaper. The user can grasp this area 9 between his fingers, making it possible to detach the sandpaper which is covered by biological material in its area 8 and cause it to be cut by the blade 7.

Three samples are taken in the orchard from the branches of a cherry tree infected with PNRSV. One sample is taken from a one year old branch by a supported rectilinear movement, applied transversely to the direction of the plant fibers. The rubbing movement against the branch should continue until the heartwood of the branch appears. At this time, fragments of vascular tissue are retained on the sandpaper (also known as emery paper).

After each sampling, the sandpaper 2 loaded with cherry tree tissue is detached from the casing 3 by means of the blade 7, as described above.

It is enough to leave this loaded sandpaper in the surrounding air for a few moments in order for dehydration to occur. The subcortical tissue fragments taken from the cherry tree branch quickly dry out on the sandpaper and remain quite green.

The user can then take each sample collected by the area 9 on the sand paper 2 and place it, as shown in FIG. 3, into one of the compartments 10 of a transport pouch 11 provided for this purpose. Once filled with samples, the pouches 11 are closed by folding back their upper part, which is provided with adhesive paper 12, protected by a paper strip (not shown), which is removed just before closing the pouch.

As seen in FIG. 3, once the pouch 11 is closed, the surface facing the user has areas allowing for a precise identification of the samples by distinctive signs, which can be handwritten notations regarding the sampling date, the requestor of the assay, the type of assay, the origin and number of samples, or also a bar code. In this way, the origin of the sample can be traced. The pouch can then be inserted in a postal envelope for mailing to the laboratory.

After reception at the laboratory, the sandpaper 2 loaded with the plant material being assayed is carefully removed from the pouch 11 by their area 9 and placed individually at the bottom of a 15 ml test tube of with a screw cap, containing 1.5 ml of extraction buffer SCPAP (as described in Minsavage et al., 1994. Development of a polymerase chain reaction protocol for detection of Xylella fastidiosa in plant tissue. Phytopathology 84:138-142).

Each tube is vortex agitated for 30 seconds to liberate the tissue fragments present on the sandpaper and then allowed to incubate at 4° C. for 10 minutes. 500 μl of the solution is collected in a 1.5 ml Eppendorf tube and centrifuged at 10,000 rpm for 5 minutes. 10 μl of the clarified (or supernatant) solution is recovered and diluted in 990 μl of distilled water.

2 μl of the diluted solution is added to 23 μl of the RT-PCR mix prepared from the Kit TITAN One tube RT-PCR (Roche) following the manufacturer's instructions, to which has been added 0.5 μl of each primer PNRSV 10F (TTC TTG MG GAC CAA CCG AGA GG (SEQ ID NO. 1)) and PNRSV 10R (GCT MC GCA GGT AAG ATT TCC AAG C (SEQ ID NO. 2)) at 20 μM. The tubes are then subjected to the RT-PCR reaction on a Thermocycler Mastercycler (Eppendorf) according to a cycle of 30 minutes at 50° C., 5 minutes at 94° C., 30 seconds at 94° C., 45 seconds at 55° C., 1 minute at 72° C. (these latter three steps are repeated 35 times) and then 10 minutes at 72° C.

The amplification products are revealed on a 1.5% agarose gel with ethidium bromide stain. The specific bands for 348 bp are present for the 3 samples taken.

The samples can be assayed directly after their arrival at the laboratory, or they can be kept in their pouch 11 at room temperature for later assaying.

### Example 2

### Detection of Virus PNRSV (Prunus necrotic Ringspot Virus) in Various Branches of Infected Cherry Trees by a RT-PCR on Purified Total Nucleic Acids (TNA)

Acquiring the samples is done the same way as in example 1 above.

On the other hand, an extraction and purification protocol for the total nucleic acids is applied according to S. Spiegel, S. W. Scott, V. Bowman-Vance, Y. Tam, N. N. Galiakparov, and A. Rosner 1996. Improved detection of Prunus necrotic ringspot virus by the polymerase chain reaction. Eur. J. Pl. Pathol. 102:681-685. The sandpaper loaded with plant material is placed in a 15 ml Falcon tube containing 2 ml of the described extraction buffer. After vigorous agitation for 1 minute at 4° C., 500 μl of the extracted solution is recovered for an extraction according to the protocol as described by Spiegel et al., 1996.

For comparison, an identical extraction of total nucleic acids is likewise done by a classical sampling protocol, namely, cutting off branches in the field with pruning shears.

To guarantee the validity of the test, one selects the same branches as those on which the sampling was already done using the invention's sampling device 1.

These branches are brought to the laboratory, where the bark is removed from each of them for around 5 cm of length, using a No. 3 scalpel blade for this. In the same area, using a No. 4 scalpel blade, the vascular tissues are scraped down to the heartwood to obtain around 400 mg of this tissue. This is collected, weighed, and put into a plastic grinding pouch, provided with a nylon net on the inside, with the addition of 10 times the volume of extraction buffer described by Spiegel et al., 1996 (that is, 4 ml for 400 mg of tissue). The grinding is done by means of a Holmex type ball homogenizer until the tissues are completely destructurized. 500 μl of the maceration is recovered for extraction by the described protocol. All these steps of bark removal, scraping, collecting, weighing and grinding should take place as quickly as possible, keeping the sampled tissues constantly at a low temperature (1 to 2° C.).

After the extraction steps, the optical density of the nucleic acid solutions obtained from the two ways of sampling is measured in the spectrophotometer (LKB Biochrom Ultrospec II, UK) at 260 nm and 280 nm in order to determine the concentration of the solutions and the degree of purity.

As can be seen in Table 1 below, the samples taken with the invention's sampling device 1 yield concentrations of total nucleic acids (TNA) of 215 μg/ml, 72 μg/ml and 222 μg/ml with respective degrees of purity of 1.34, 1.55 and 1.34. The samples taken in the classical manner yield concentrations of total nucleic acids of 138 μg/ml, 119 μg/ml and 86 μg/ml with respective degrees of purity of 1.56, 1.36 and 1.48.

TABLE 1

| | | | | | Branch 3 | |
|---|---|---|---|---|---|---|
| | Branch 1 | | Branch 2 | | | Degree |
| | TNA | Degree | TNA | Degree | TNA | of |
| | (μg/ml) | of purity | (μg/ml) | of purity | (μg/ml) | purity |
| Invention | 215 | 1.34 | 72 | 1.55 | 222 | 1.34 |
| Classical | 138 | 1.56 | 119 | 1.36 | 86 | 1.48 |
| method | | | | | | |

The results obtained show that the sampling done according to the invention is able to produce a concentration of total nucleic acids comparable to that of the classical technique, and that the quality of the total nucleic acids extracted is similar in terms of degree of purity.

For the amplification reaction, 2 μl of the total nucleic acids, diluted 100×, are added to the 23 μl of the RT-PCR mix prepared from the Kit TITAN One tube RT-PCR (Roche) and following the directions of the manufacturer, to which was added 0.5 μl of each primer PNRSV 10F (TTC TTG MG GAC CM CCG AGA GG (SEQ ID NO. 1)) and PNRSV 10R (GCT MC GCA GGT MG ATT TCC MG C (SEQ ID NO. 2)) at 20 μM. The tubes are then subjected to the RT-PCR reaction on a Thermocycler Mastercycler (Eppendorf) for a cycle of 30 minutes at 50° C., 5 minutes at 94° C., 30 seconds at 94° C., 45 seconds at 55° C., 1 minute at 72° C. (these latter three steps are repeated 35 times) and then 10 minutes at 72° C.

The amplification products are revealed on a 1.5% agarose gel with ethidium bromide stain. The specific bands for 348 bp are presented both for the 3 samples taken according to the invention and for the 3 classical samples.

### Example 3

Detection by Real-Time PCR of the Virus BSV on the Banana from Samples Taken at the Main Leaf Rib

As can be seen in FIG. 4, in this sample embodiment of the invention one uses a sampling device 13 more particularly adapted to the taking of deep tissue samples.

The device 13 is comprised of a rigid plastic or metal stylet 14, 4 cm in length and 3 mm in diameter, one of whose ends is formed by a cone 15, 0.5 cm in diameter at the base and 1 cm in length. The cone 15 has asperities 16 in the form of pointed scoops, 1 mm in height, distributed over its surface.

The stylet 14 is screwed onto the spindle of a micro drill 15. The sampling is done by rotation at very low speed (500 rpm) of the stylet 14, which penetrates the main rib 18 of the banana leaf 19. The rotation lasts around 1 to 2 seconds.

The stylet 14, loaded with plant material, is then recovered, freed of excess tissue, allowed to dry for a few moments in the surrounding air. A second sampling is done in the same way with a second stylet. The stylets are placed in a pouch of the type 11 shown in FIG. 3 and left at room temperature prior to their transport to the laboratory.

In parallel and for purposes of comparison, a piece 5 cm in length is cut out by scalpel from the main rib of the same leaf 19 and immediately frozen to await its transport to the laboratory.

Once received at the laboratory, one of the two stylets loaded with plant material is placed in a 15 ml Falcon tube containing 3 ml of extraction buffer (137 mM NaCl, 8 mM Na₂HPO₄, 1.5 mM KH₂PO₄, 2.7 mM KCl, 3 mM NaN₃, 0.05% Tween 20, and 80 mM Na₂SO₃, pH 7.2 to 7.4).

The second stylet is kept in the pouch at room temperature for four weeks, to be assayed later on.

The Falcon tube is vortex agitated for 1 minute to free the tissue fragments and then allowed to incubate at 4° C. for 5 minutes. 500 μl of solution is collected in a 1.5 ml Eppendorf tube and centrifuged at 7000 rpm for 10 minutes. 100 μl of the clarified supernatant is recovered and diluted in 900 μl of distilled water.

1 μl of the diluted solution is added to 49 μl of the PCR mix prepared from 0.2 mM of each dNTP, 1 unit of Taq DNA polymerase (Roche), 1.4×PCR buffer, 2 mM MgCl2 (final concentration), 0.28 μM of each primer and 0.1 μM of the MGB probes, as defined in M. Delanoy, M. Salmon, and J. Kummert, 2003. Development of Real-Time PCR for the Rapid Detection of Episomal Banana streak virus (BSV). Plant disease 87:33-38. The tubes are then subjected to the PCR reaction with detection in real time in a Thermocycler GeneAmp 5700 Sequence Detection (Applied Biosystems) according to a cycle of 1 minute at 95° C., 30 seconds at 95° C., 20 seconds at 53° C., 1 minute at 60° C. (these latter three steps are repeated 50 times).

The intensity of fluorescence measured during the cycles is plotted on a graph and the value of the Ct (cycle at which the intensity of the fluorescence begins to go above the threshold) is determined.

Comparative tests were performed between a classical sampling (grinding of 400 mg of frozen banana leaf ribs in 4 ml of the same extraction buffer by means of a Holmex homogenizer) and example 2 of the invention, as described above.

Furthermore, four weeks after this first assay, a new preparation of the crude extract was made in the same way for the second stylet, which had been kept at room temperature in the pouch, and for 400 mg of frozen leaf rib.

As shown in Table 2 below, the results for the classical preparation and the invention are shown, respectively, by the Ct values (Cycle threshold corresponding to a threshold of 0.025) of 35 and of 30 on the day of the sampling and 35 and 31 four weeks after the sampling. The final fluorescence values are respectively 0.21 and 0.30 the day of the sampling and 0.19 and 0.28 four weeks after the sampling.

TABLE 2

| | Classical preparation | | Invention | |
|---|---|---|---|---|
| | Ct (threshold | Final | Ct (threshold | Final |
| | 0.025) | fluorescence | 0.025) | fluorescence |
| Assay on the | 35 | 0.21 | 30 | 0.30 |
| day of | | | | |
| sampling | | | | |
| Assay 4 weeks | 35 | 0.19 | 31 | 0.28 |
| after sampling | | | | |

One thus finds that the invention can produce an earlier fluorescence signal and a higher final value than with the classical technique. What is more, the results obtained with the material prepared four weeks in advance are just as good as those obtained the day of the sampling.

### Example 4

### Detection by RT-PCR of the Virus ASGV (Apple Stem Grooving Virus) on Apple Tree Branches Received as Fresh Material at the Laboratory

This example shows how the method of assaying nucleic acids by molecular hybridization according to the invention is specifically applicable to a phytopathology diagnostic laboratory. The sampling device used here consists of a simple rectangle of 1.5×3 cm cut out from sandpaper GUMIC P 100.

In fact, the working conditions are such that it is not necessary to use a distributor, or to penetrate into the deep tissue layers. Thus, a simplified form of the sampling device according to the invention is sufficient.

The branch is placed on a work table in front of the operator, who holds its end, the sandpaper is held beneath the index finger of the free hand of the operator, who applies rubbing by slight pressure transversely to the lengthwise axis of the branch. The rubbing is continued until the heartwood is reached. This sample corresponds to around 300 mg of tissue. The sandpaper loaded with plant tissue is then placed at the bottom of a 15 ml glass tube with a screw cap, containing 1 ml of extraction buffer TE at 4° C. (50 mM Tris, pH 8.0, 10 mM EDTA). All the branches are prepared in the same way.

The tube is vortex agitated for 30 seconds to release the tissue fragments and then allowed to incubate at 4° C. for 10 minutes. 500 μl of solution is recovered in a 1.5 ml Eppendorf tube and centrifuged at 10,000 rpm for 5 minutes. 10 μl of the clarified supernatant is recovered and diluted in 990 μl of distilled water.

2 μl of the diluted solution is added to the 23 μl of the RT-PCR mix prepared from the Kit TITAN One tube RT-PCR (Roche) and following the manufacturer's directions, to which was added 0.5 μl of each primer at 20 μM (ASGV5F and ASGV5R), as defined in J. Kummert, M. Vendrame, S. Steyer, and P. Lepoivre, 2000. Development of routine RT-PCR tests for certification of fruit tree multiplication material. EPPO Bulletin 30: 441-448.

The tubes are then subjected to the RT-PCR reaction in a Thermocycler Mastercycler (Eppendorf) according to a cycle of 30 minutes at 50° C., 5 minutes at 94° C., 30 seconds at 94° C., 45 seconds at 55° C., 1 minute at 72° C. (these latter three steps are repeated 35 times) and then 10 minutes at 72° C.

The amplification products are revealed on a 1.5% agarose gel with ethidium bromide stain. The specific band is situated at a level of 344 bp.

One finds that these results are just as convincing as those done with a classical method, starting from fresh plant material.

As one can discover from the foregoing, the method of assaying according to the invention, thanks to the stage of taking samples of biological material with abrasive sampling means, limits the effects of oxidation very efficiently. The target nucleic acids present in the plant tissues sampled do not need to be placed in direct contact with a buffered liquid medium (unlike the case when working with fresh tissues).

The method per the invention requires only a sampling of 20 to 40 mg of biological material, or 5 to 10 times less than the classical technique.

It should be noted that, while the method of the invention allows one to work on crude extract in the laboratory, one would still be within its scope, of course, if additional purification steps were carried out.

As for the samples, these can be assayed one by one, or grouped together for a common assay, depending on the goals required.

1. A method of assaying nucleic acids by molecular hybridization, which comprises:
taking samples of biological material by a sampling device comprising abrasive sampling means capable of retaining biological material in the form of cells;
isolating nucleic acids from the cells; and
assaying the nucleic acids by molecular hybridization.

2. The method according to claim 1, wherein the sampling of biological material is done in the surrounding air.

3. The method according to claim 1, wherein the sampling is done outside of a laboratory where the assaying will be done, and further comprising transporting the abrasive sampling means loaded with their respective samples of biological material to said laboratory.

4. The method according to claim 1, further comprising extraction of the nucleic acids, comprising the steps of:
immersing the abrasive sampling means loaded with their respective samples of biological material into an extraction buffer,
agitating the extraction buffers,
separating the nucleic acids, and
recovering clarified solution containing the nucleic acids.

5. The method according to claim 4, wherein the separation step consists of a centrifugation, and the supernatant constitutes the clarified solution.

6. The method according to claim 1, wherein the assaying by molecular hybridization is done by polymerase chain reaction (PCR).

7. The method according to claim 1, further comprising determining the presence of a pathogenic agent in the biological material by the molecular hybridization.

8. The method according to claim 1, wherein the biological material consists of material of plant origin.

9. A kit for implementing the method according to claim 1 which comprises a sampling device comprising abrasive sampling means able to retain biological material in the form of cells.

10. The kit according to claim 9, wherein the sampling means comprise a solid material comprising an abrasive outer surface.

11. The kit according to claim 10, wherein the solid material is selected from the group consisting of silica, glass, metals, carbon fibers and plastics.

12. The kit according to claim 10, wherein the abrasive outer surface comprises hardness capable of retaining cells of biological material.

13. The kit according to claim 9, wherein the sampling device comprises a support able to support the abrasive sampling means.

14. The kit according to claim 9, further comprising means for the transport of the abrasive sampling means.

15. The kit according to claim 9, further comprising means of identification of the abrasive sampling means.

16. The kit according to claim 9, further comprising extraction buffer for assaying nucleic acids by hybridization.

17. The kit according to claim 9, further comprising specific reagents of PCR reactions.
